(19) United States
(12) Patent Application Publication
(10) Pub. No.: US 2004/0116795 A1
(43) Pub. Date: Jun. 17, 2004

(54) Determination of dose-enhancing agent concentration and dose enhancement ratio

(76) Inventors: William F. Collins, Clayton, CA (US); Michael D. Weil, Fort Collins, CO (US)

Correspondence Address: Siemens Corporation, Attn: Elsa Keller, Legal Administrator, Intellectual Property Department, 186 Wood Avenue South, Iselin, NJ, 08830, US

(21) Appl. No.: 10/320,906

(22) Filed: Dec. 17, 2002

(51) Int. Cl. A61B 5/05

(52) U.S. Cl. 600/407; 600/427

(57) ABSTRACT

A system includes a surface to support a body and a CT scanner to acquire, based on a first set of one or more CT scanning parameters, a first set of CT data representing a volume within the body including a dose-enhancing agent. The system further includes a processor to determine a CT number associated with the volume based on the first set of CT data, and to determine a dose enhancement corresponding to the dose-enhancing agent within the volume based on a stored association between the CT number, the first set of one or more scanning parameters, and the dose enhancement.

### BACKGROUND

1. Field

The present invention relates generally to medical treatment using a dose-enhancing agent, and more particularly to systems to determine a concentration of such an agent within a volume and/or a dose enhancement ratio attributable to the agent.

2. Description

During radiation treatment, a radiation beam is directed at a tumor located within a patient. The radiation beam is intended to deliver a predetermined dose of treatment radiation to the tumor according to a treatment plan. The goal of such treatment is to kill tumor cells through ionizations caused by the radiation.

A kilovoltage radiation treatment system such as those described in U.S. Pat. No. 6,366,801 to Cash et al produces a divergent beam of traditional medical x-rays having energies in the 50 to 150 keV range and focuses the beam on a target site using a lens designed for this purpose. At these energies, most cellular damage caused by the radiation beam is due to photoelectric absorption. This absorption, and the resulting cellular damage, may be magnified by injecting a heavy element-carrying biochemical agent into the target site.

In the kilovoltage energy range, the absorption cross-section of an element having an atomic weight greater than 50 is often significantly higher than elements of which most human tissue is composed. Therefore, if a suitable radiation beam irradiates a tissue volume containing such an element, more photons will be stopped by the volume than in the absence of the element. The resulting tissue damage will be greater than tissue damage that would occur without the element, because most of the increased stoppages will be due to photoelectric absorption.

The increased cellular damage due to the presence of a heavy element is often referred to as a dose enhancement. Specifically, the dose enhancement is expressed as a ratio of the number of centiGray (cGy) experienced by the tissue including the heavy element to the number of cGy experienced in adjacent tissue that is substantially free from the heavy element. Accordingly, any heavy element-carrying biochemical agent will be referred to herein as a dose-enhancing agent.

The dose-enhancing effect of dose-enhancing agents can be beneficial, since cure rates for tumors often increase with increased radiation doses. If a dose-enhancing agent is used in conjunction with a treatment plan, however, the dispersion and the concentration of the agent are monitored in order to ensure that particular tissues experience particular doses as specified by the treatment plan. Cash describes systems to monitor a location of an injected agent and to determine a dose enhancement resulting from the injected agent, but superior systems are desired.

### SUMMARY

To address at least the foregoing, some embodiments of the present invention provide a system, method, apparatus, and means to determine a CT number corresponding to each of a plurality of dose enhancements corresponding to a dose-enhancing agent for each of a plurality of sets of one or more CT scanning parameters. In further aspects, some embodiments include acquisition of a set of CT data representing a volume within a body, the volume including the dose-enhancing agent, determination of a CT number associated with the volume based on the set of CT data, and determination of a dose enhancement corresponding to the dose-enhancing agent within the volume based on the CT number associated with the volume and on the CT number corresponding to each of the plurality of dose enhancements for each of the plurality of sets of one or more CT scanning parameters.

In another aspect, provided are acquisition of a first set of CT data representing a phantom, the phantom comprising a dose-enhancing agent having a sample concentration, and association of a CT number from the first set of CT data with the sample concentration of the dose-enhancing agent. Further aspects provide acquisition of a second set of CT data representing the phantom in conjunction with a second set of one or more scanning parameters, the second set being different from the first set, and association of a CT number from the second set of CT data with the sample concentration of the dose-enhancing agent and with one or more of the second set of one or more scanning parameters.

In still another aspect, some embodiments provide acquisition, based on a first set of one or more CT scanning parameters, of a first set of CT data representing a volume within a body including a dose-enhancing agent, determination of a CT number associated with the volume based on the first set of CT data, and determination of a dose enhancement corresponding to the dose-enhancing agent within the volume based on an association between the CT number, the first set of one or more scanning parameters, and the dose enhancement.

The present invention is not limited to the disclosed embodiments, however, as those of ordinary skill in the art can readily adapt the teachings herein to create other embodiments and applications.

### BRIEF DESCRIPTION OF THE DRAWINGS

The exact nature of this invention, as well as its objects and advantages, will become readily apparent from consideration of the following specification as illustrated in the accompanying drawings, in which like reference numerals designate like parts, and wherein:

FIG. 1 is a diagram illustrating a radiation treatment room according to some embodiments of the present invention;

FIG. 2 is a block diagram illustrating elements of a radiation treatment system according to some embodiments of the present invention;

FIGS. 3a and 3b are perspective views of a phantom for use in conjunction with some embodiments of the present invention;

FIG. 4 is a flow diagram of process steps according to some embodiments of the present invention;

FIG. 5 is a representative view of a portion of a data table according to some embodiments of the present invention;

FIG. 6 is a flow diagram of process steps according to some embodiments of the present invention;

FIG. 7 is a flow diagram of process steps according to some embodiments of the present invention;

FIG. 8 is a computed tomography (CT) image according to some embodiments of the present invention;

FIG. 9 is a CT image according to some embodiments of the present invention; and

FIG. 10 is a CT image according to some embodiments of the present invention.

### DETAILED DESCRIPTION

The following description is provided to enable any person of ordinary skill in the art to make and use the invention and sets forth the best modes contemplated by the inventor for carrying out the invention. Various modifications, however, will remain readily apparent to those in the art.

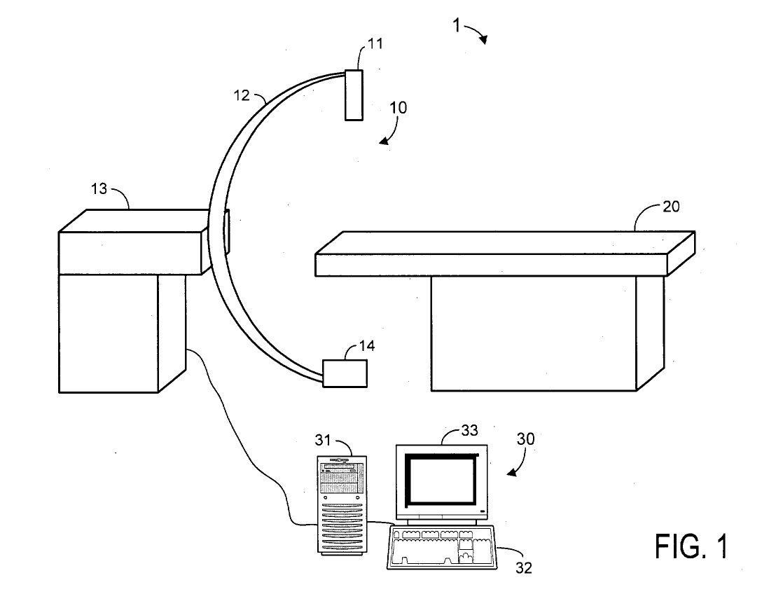

FIG. 1 illustrates radiology room 1 pursuant to some embodiments of the present invention. Radiology room 1 includes radiation unit 10, treatment table 20 and operator station 30. The elements of radiology room 1 may be used to acquire CT data, to determine a treatment plan and/or to deliver radiation to a patient according to a treatment plan.

Radiation unit 10 includes treatment head 11, c-arm 12, base 13 and imaging system 14. Treatment head 11 includes a beam-emitting device such as an x-ray tube for emitting radiation used during calibration, data acquisition and/or treatment. The radiation may comprise electron, photon or any other type of radiation, and may have energies ranging from 50 to 150 keV. The radiation emitted by treatment head 11 may comprise any radiation suitable for data acquisition and/or treatment according to some embodiments of the present invention. In some embodiments, the radiation is suitable to produce dose-enhancing effects when used in conjunction with a heavy element-carrying biochemical agent.

Treatment head 11 also includes a cylinder in which are disposed optics such as a focusing lens for optically processing the emitted radiation. The focusing lens may comprise a lens for producing a convergent radiation beam from radiation emitted by the x-ray tube. Examples of this type of lens are described in U.S. Pat. No. 6,359,963 to Cash, in U.S. Pat. No. 5,604,782 to Cash, Jr., in U.S. patent application Publication No. 2001/0043667 of Antonell et al., and/or elsewhere in currently or hereafter-known art. Treatment head 11 may also include beam-shaping devices such as one or more jaws, collimators, reticles and apertures.

C-arm 12 is slidably mounted on base 13 and can therefore be moved in order to change the position of treatment head 11 with respect to table 20. In some embodiments, base 13 also includes a high-voltage generator for supplying power used by treatment head 11 to generate kilovoltage radiation. Many c-arm/base configurations may be used in conjunction with some embodiments of the present invention, including configurations in which base 13 is rotatably mounted to a ceiling of room 1, configurations in which one c-arm is slidably mounted on another c-arm, and configurations incorporating multiple independent c-arms. In some embodiments, the configuration allows acquisition of CT data.

Examples of c-arm kilovoltage radiation units include Siemens SIREMOBIL™, ISO-C30™, MULTISTAR™, BICOR™ and POLYSTAR™ units as well as other units designed to perform tomography and/or angiography. These units are often less bulky and less costly than radiation systems that utilize radiation energies in the megavoltage range. Of course, any system(s) for acquiring CT data and/or delivering treatment radiation may be used in conjunction with some embodiments of the present invention.

Imaging system 14 may comprise an image intensifier and a camera. An image intensifier is a vacuum tube that converts x-rays to visible light, which is then detected by the camera to produce an image. Imaging system 14 may also comprise a flat-panel imaging system that uses a scintillator and silicon sensor elements or direct x-ray conversion detectors to produce an image based on received radiation.

Imaging system 14 acquires CT data representing the attenuative properties of material that is located between treatment head 11 and imaging system 14 while radiation is emitted from treatment head 11. CT data is often represented in Hounsfield units (HU), or CT numbers. In order to acquire this data, imager measures x-ray radiation emitted by treatment head 11 after it passes through the material and converts it to an electrical signal. The electrical signal is then converted to digital data.

Each slice of the material is divided into small cubes, and each cube is compressed into a 2-dimensional square and assigned a CT number. In a case that the CT number is expressed in HU, the number represents the radiation density of a square of the material relative to water. The CT data may be used to generate a visual representation of the relative densities of each square of material.

The CT data is acquired in accordance with particular scan parameters. The parameters may include x-ray tube current, x-ray tube potential, radiation energy, scan time, slice width and radiation filtration parameters. The parameters are often established so as to produce CT data having ranges from +1000 HU to −1000 HU. +1000 HU, 0 HU and −1000 HU are intended to represent, respectively, the radiation density of cortical bone, water, and air. Images based on this data commonly represent +1000 HU with white pixels, 0 HU with central gray pixels, and −1000 HU with black pixels. Of course, other CT data formats and scales may be used in conjunction with some embodiments, and images may be generated from this CT data according to any suitable convention.

During CT data acquisition and/or radiation treatment, a phantom and/or a patient is placed on treatment table 20 in order to position a region of interest between treatment head 11 and imaging system 14. Accordingly, table 20 may comprise mechanical systems for moving itself with respect to unit 10.

Operator station 30 includes a tower 31 in communication with an input device such as keyboard 32 and an operator display 33. An operator may operate operator station 30 to acquire CT data, to plan radiation treatment, and/or to deliver radiation treatment according to some embodiments of the invention. Operator station 30 may be located apart from radiation unit 10, such as in a different room, in order to protect the operator from radiation. It should be noted, however, that the operation of low-voltage radiation systems does not require protective measures to the extent of those taken during megavoltage radiation therapy, resulting in less costly therapy.

Tower 31 may store processor-executable process steps according to some embodiments of the present invention. In some aspects, the process steps are executed by operator station 30, radiation unit 10, and/or another device to determine a CT number corresponding to each of a plurality of concentrations of a dose-enhancing agent for each of a plurality of sets of one or more CT scanning parameters.

The process steps may also provide acquisition of a first set of CT data representing a phantom, the phantom comprising a dose-enhancing agent having a sample concentration, association of a CT number from the first set of CT data with the sample concentration of the dose-enhancing agent, acquisition of a second set of CT data representing the phantom in conjunction with a second set of one or more scanning parameters, the second set being different from the first set, and association of a CT number from the second set of CT data with the sample concentration of the dose-enhancing agent and with one or more of the second set of one or more scanning parameters.

Process steps according to some embodiments provide acquisition, based on a first set of one or more CT scanning parameters, of a first set of CT data representing a volume within a body including a dose-enhancing agent, determination of a CT number associated with the volume based on the first set of CT data, and determination of a concentration of the dose-enhancing agent within the volume based on the CT number associated with the volume and on an association between a second CT number, a second set of one or more scanning parameters, and a second concentration of the dose-enhancing agent.

The above-described steps may also be embodied, in whole or in part, by hardware of tower 31, radiation unit 10, and another device. In some embodiments, the process steps are not performed by a single device, but are performed by a data acquisition device and a separate radiation treatment device. Of course, the environment shown in FIG. 1 may include less or more elements than those shown. In addition, embodiments of the invention are not limited to the devices and/or the environment shown.

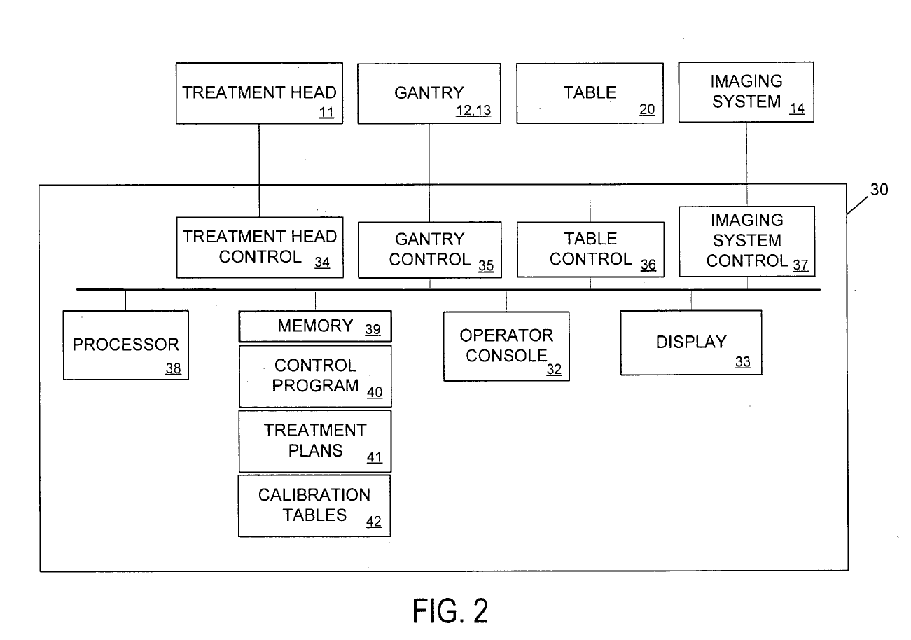

FIG. 2 is a block diagram of elements of radiology room 1 according to some embodiments. As shown, operator station 30 includes several elements for interfacing with other elements of room 1. Specifically, operator station includes treatment head control 34, gantry control 35, table control 36, imager control 37, processor 38 and memory 39.

Treatment head control 34 controls treatment head 11 so as to implement particular scanning parameters related to treatment head 11. These parameters may include an x-ray tube potential, a radiation energy, an x-ray tube current, a scan time and radiation filtration parameters. Gantry control 35, table control 36 and imaging system control 37 also operate to control c-arm 12, base 13, table 20 and imaging system 14 in accordance with acquisition and/or treatment parameters.

Processor 38 executes processor-executable process steps stored in memory 39. In this regard, memory 39 stores processor-executable process steps of control program 40. These steps may be executed to provide operation according to some embodiments. Memory 39 may also store treatment plans 41 and calibration tables 42. Treatment plans 41 may comprise scripts that are automatically executable by radiation unit 10 and treatment table 20 in order to provide data acquisition and/or multiple treatment segments. Treatment plans 41 may also comprise any other currently- or hereafter-known types of treatment plan.

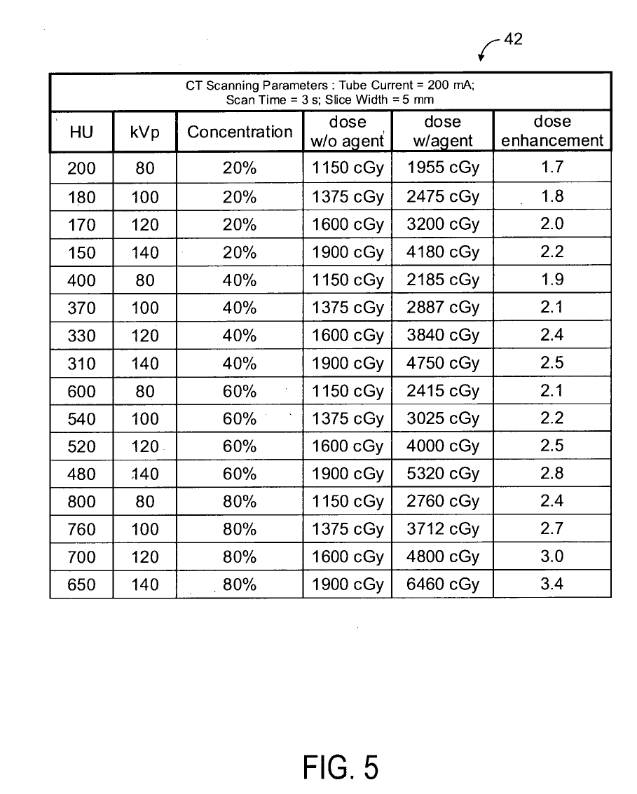

Calibration tables 42 may associate CT numbers with concentrations of dose-enhancing agents. The associations may correspond to particular scanning parameters, and may be used to determine a concentration of and/or a dose enhancement effect resulting from a dose-enhancing agent introduced into a body. Calibration tables 42 may also or alternatively associate CT numbers with dose enhancement ratios for particular scanning parameters, and will be described in more detail below with respect to FIG. 5.

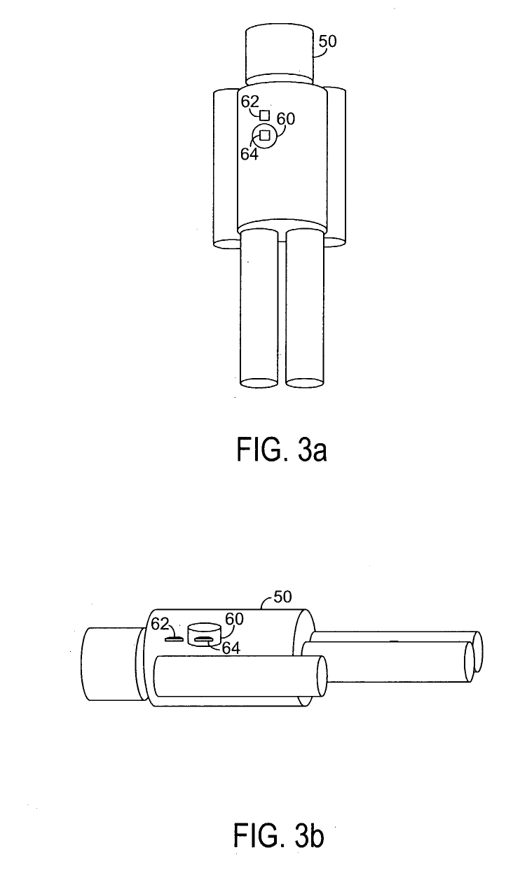

FIGS. 3a and 3b are representative views of phantom 50 according to some embodiments. Phantom 50 may be composed of materials that simulate the radiation density profile of a human body. Phantom 50 may also allow volumes of material to be placed at various locations therein.

For example, placed within phantom 50 is agent sample 60, which may be enclosed in any suitable container. Agent sample 60 may include a known concentration of any dose-enhancing agent. The dose-enhancing agent may be composed of any currently- or hereafter-known dose-enhancing materials, including Iodine, Gadolinium, and Gold. As will be described below, phantom 50 and agent sample 60 are used to generate calibration tables 42 according to some embodiments.

Phantom 50 also includes spaces into which film dosimeters such as dosimeters 62 and 64 may be placed. Film dosimeters 62 and 64 may comprise special film dosimeters based on kilovoltage x-ray energy radio-chromic films sensitive to short-range (10 to 50 microns) photoelectrons (example: GAFCHROMIC type HD 810). In the illustrated embodiment, film dosimeter 62 is placed adjacent to agent sample 60 and film dosimeter 64 is placed within agent sample 60. Such an arrangement allows determination of a dose enhancement corresponding to agent sample 60 according to some embodiments.

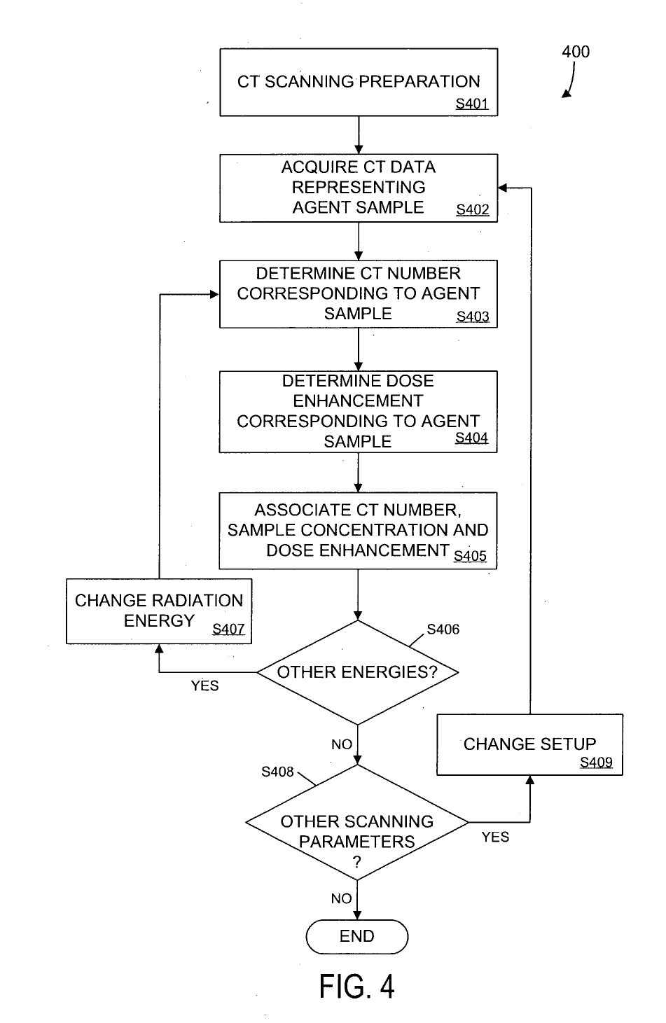

FIG. 4 comprises a flow diagram of process steps 400 to generate calibration tables 42 according to some embodiments of the invention. Process steps 400 may be embodied by hardware, firmware and/or software of tower 31, radiation unit 10, and/or another device.

Preparation for CT scanning commences in step S401. Preparation may include establishing particular CT scanning parameters using treatment head control 34, gantry control 35, table control 36, and imager control 37. According to the present example, the x-ray tube current is set to 200 mA, the scan time is set to 3 s, and the slice width is set to 5 mm. In addition, the energy of emitted radiation is initially set to 120 keV by setting a potential of the x-ray tube of treatment head 11 to 120 kVp.

Agent sample 60 is placed in phantom 50 in step S401. More particularly, a container including a known concentration of sample 60 may be placed inside phantom 50, or the known concentration may be injected into a volume within phantom 50. In the present example, the concentration of the agent is 20% by weight. According to some embodiments, agent sample 60 is permanently mounted within phantom 50.

Film dosimeters 62 and 64 may also be placed within phantom 50 in step S401. In one example, film dosimeter 64 is placed within a cavity of a container holding agent sample 60. One or both of film dosimeters may be fixedly mounted within phantom 50. Phantom 50 is also positioned on table 20 in step S401 so as to allow for acquisition of CT data representing sample 60.

Next, in step S402, phantom 50 is scanned according to the established scanning parameters so as to acquire CT data representing agent sample 60. In some embodiments, scanning produces CT numbers corresponding to two-dimensional slices of phantom 50. A CT number corresponding to the agent sample is determined from the CT data in step S403. It will be assumed that the determined CT number is 170 HU.

A dose enhancement corresponding to agent sample 60 may be determined in step S404 by determining a dose experienced by each of film dosimeters 62 and 64. The dose experienced by dosimeter 64 is divided by the dose experienced by dosimeter 62 to determine the dose enhancement. Other methods for determining the dose enhancement attributable to agent sample 60 may be used in conjunction with some embodiments of the present invention.

The determined CT number is then associated with the known concentration of the sample in step S405. Also associated with the CT number may be the dose enhancement determined in step S404. According to some embodiments of step S405, these values are associated with one another by storing the values in associated fields of calibration tables 42.

FIG. 5 is a tabular representation of a portion of calibration tables 42 according to some embodiments. The portion specifies the CT scanning parameters established in step S401, and includes four columns for associating respective values of HU, kVp, concentration, dose without the agent (experienced by dosimeter 62), dose with the agent (experienced by dosimeter 64), and dose enhancement. As shown, the third row of data of table 42 associates the determined CT number and the known concentration of the present example.

Next, in step S406, it is determined whether calibration information for additional energies is desired. If so, flow proceeds to step S407 in which the energy of the scanning radiation is changed. Flow returns to step S403 from step S407 and continues as described above until calibration information for each desired radiation energy has been obtained. FIG. 5 shows that calibration information for an additional three x-ray tube potentials (80 kVp, 100 kVp and 140 kVp) was obtained and stored in step S405.

Flow continues to step S407 if it is determined in step S405 that calibration information for additional energies is not desired. In step S407, it is determined whether calibration information for other CT scanning parameters is desired. If so, the CT scanning parameters are changed in step S409 and flow returns to step S402. Any new calibration information obtained for the changed scanning parameters may also be stored in calibration tables 42. However, since the portion illustrated in FIG. 5 is associated with the original parameters, the new calibration information is stored in other portions of calibration tables 42. Process steps 400 terminate once the determination of step S408 is negative.

FIG. 5 shows calibration information that associates CT numbers with agent concentrations other than 20%. Such information may be obtained as described above, although by using agent samples having the other agent concentrations. In addition, a dose enhancement associated with particular scanning parameters and a particular concentration may be determined by performing a first scan while a dosimeter is in a first location, and performing a second scan while an agent sample containing a dosimeter is in the first location. Of course, calibration tables 42 may be structured differently than as shown in FIG. 5, and may include more or less information than that shown.

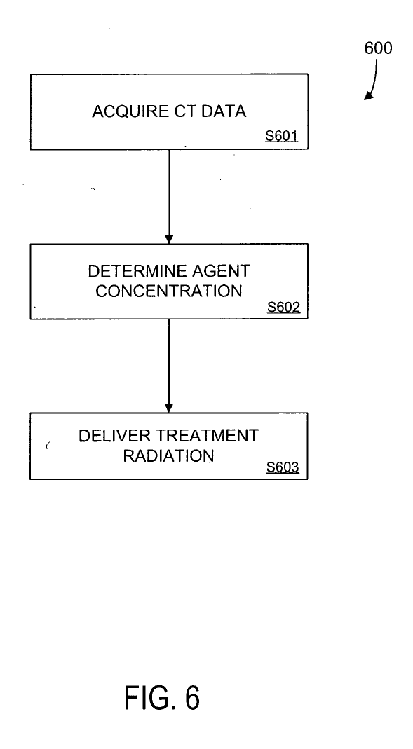

FIG. 6 is a flow diagram of process steps 600 according to some embodiments. Process steps 600 may be used to determine the concentration of a dose-enhancing agent within a body. Process steps 600 may be performed by a device that was used to perform process steps 400 or by a different device.

Initially, prior to step S601, a patient is positioned on table 20 in accordance with a treatment plan. A dose-enhancing agent is then introduced into a region of interest within the patient. The agent may be introduced via direct injection, intravenous injection, or by other means. CT data representing the region of interest is then acquired in step S601. The CT data is acquired according to scanning parameters specified by the treatment plan.

A concentration of the dose-enhancing agent at the region of interest is determined in step S602. According to some embodiments of step S602, a CT number corresponding to the region of interest is determined from the acquired CT data. A calibration table corresponding to the CT scanning parameters used in step S601 is identified, and a concentration associated with the CT number is determined from the table. For example, assuming that the scanning parameters are identical to those shown in the first row of the FIG. 5 table, the x-ray tube potential is 100 kVp, and the CT number is 540 HU, an agent concentration of 60% is determined from the FIG. 5 table. In a case that one or more of the scanning parameters, radiation energy and CT number are not identically specified in one of calibration tables 42, any interpolation method may be used to determine the concentration in step S602.

Process steps 600 assume that the concentration (and resulting dose enhancement) determined in step S602 is consistent with a prescribed treatment plan. Accordingly, treatment radiation is delivered according to the treatment plan in step S603. In some embodiments, the energy of the delivered treatment radiation is similar to the energy of radiation used to acquire the CT data in step S601. If the concentration determined in step S602 is not consistent with the treatment plan, the concentration may be changed by introducing additional agent into the region of interest or waiting for the body's biological processes to flush out a portion of the agent from the region of interest.

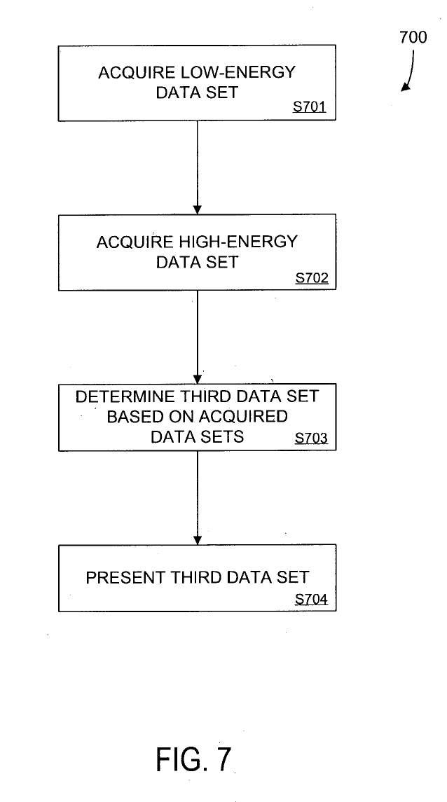

FIG. 7 is a flow diagram of process steps 700 to locate a position of dose-enhancing agent within a body. Process steps 700 may be embodied in hardware and/or software of imaging system 14, tower 31 and/or another device.

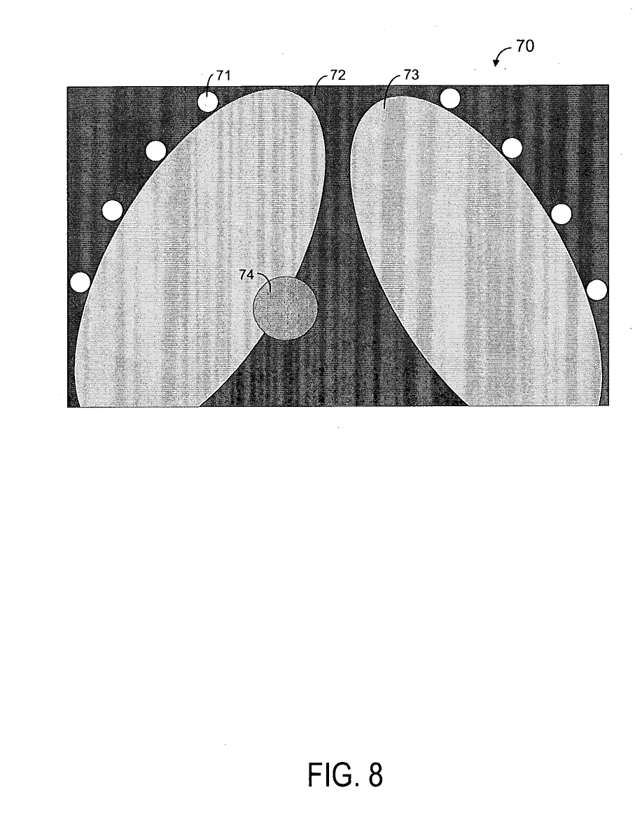

After positioning the patient on table 20 and introducing a dose-enhancing agent into a region of interest, the region of interest is scanned in step S701. The region is scanned using low-energy radiation and imaging system 14 acquires a set of CT data representing the region. FIG. 8 shows image 70 that is generated based on the acquired CT data.

Image 70 shows bone areas 71, lung areas 72, watery tissue areas 73, and agent area 74. According to image 70, areas corresponding to higher HU values are lighter than areas corresponding to lower HU values. Therefore, agent area 74 is associated with a lower HU value than lung areas 72. Other imaging conventions may be used in conjunction with some embodiments. For example, areas corresponding to higher HU values may be darker than areas corresponding to lower HU values.

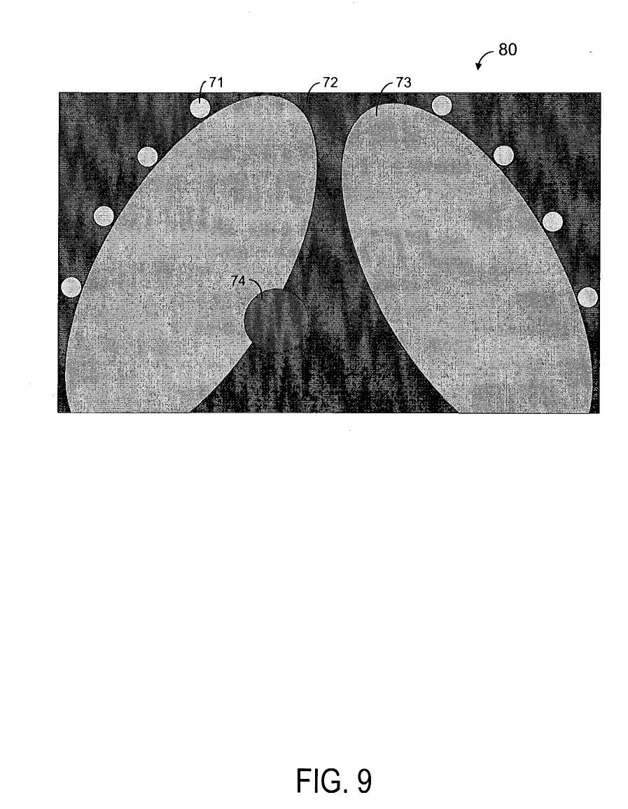

A set of CT data is acquired in step S702 using higher-energy scanning radiation than that used in step S701. FIG. 9 shows image 80 representing the data acquired in step S702. Image 80 follows the same convention as image 70, in that areas corresponding to higher HU values are lighter than areas corresponding to lower HU values. Image 80 shows that the HU values corresponding to each of areas 71 through 74 have decreased with respect to the HU values represented in image 70. However, due to the physical characteristics of the dose-enhancing agent located in area 74, the HU value associated therewith has decreased in a greater proportion than the HU values associated with the other areas.

A third data set is determined in step S703 based on the data sets acquired in steps S701 and S702. The third data set may represent a difference between the first and second data sets. More specifically, the third data set is determined according to some embodiments by subtracting a value of each data point of the second data set from a corresponding data point of the first data set. Since the HU values corresponding to area 74 decreased more from the first data set to the second data set than did the HU values of the other areas, the difference between the first and second data set is greater for data points associated with area 74. Accordingly, the location of the dose-enhancing agent may be determined by locating a region of the third data set that is associated with a larger difference in HU values than other regions.

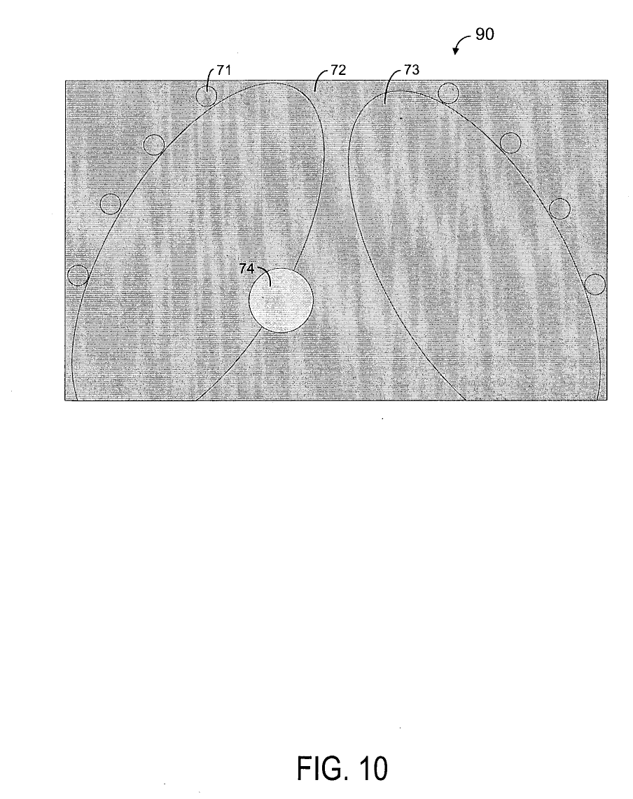

Image 90 of FIG. 10 represents the third data set, and may be presented to an operator via display 33 in step S704. Image 90 may therefore be used to confirm a location and a uniformity in the distribution of the dose-enhancing agent. According to image 90, the HU values corresponding to regions 71 through 73 decreased substantially similarly between the first data set and the second data set. In order to accentuate area 74, image 90 may use a HU-to-pixel color conversion scale that is different from that used in images 70 and 80. For example, the pixels of image 90 may range from 0 HU (black) to 50 HU (white), rather than from −1000 HU to +1000 HU.

Those in the art will appreciate that various adaptations and modifications of the above-described embodiments can be configured without departing from the scope and spirit of the invention. Therefore, it is to be understood that, within the scope of the appended claims, the invention may be practiced other than as specifically described herein.

What is claimed is:

1. A method comprising:
determining a CT number corresponding to each of a plurality of dose enhancements corresponding to a dose-enhancing agent for each of a plurality of sets of one or more CT scanning parameters.

2. A method according to claim 1, wherein the one or more CT scanning parameters comprise at least one of:
an x-ray tube potential;
a scanning radiation energy;
an x-ray tube current;
a scan time;
slice width; and
radiation filtration parameters.

3. A method according to claim 1, further comprising:
acquiring a set of CT data representing a volume within a body, the volume including the dose-enhancing agent;
determining a CT number associated with the volume based on the set of CT data; and
determining a dose enhancement corresponding to the dose-enhancing agent within the volume based on the CT number associated with the volume and on the CT number corresponding to each of the plurality of dose-enhancements for each of the plurality of sets of one or more CT scanning parameters.

4. A method according to claim 1, further comprising:
determining a concentration of the dose-enhancing agent corresponding to each of the plurality of dose enhancements for each of the plurality of sets of one or more CT scanning parameters.

5. A method comprising:
acquiring a first set of CT data representing a phantom, the phantom comprising a dose-enhancing agent having a sample concentration; and
associating a CT number from the first set of CT data with the sample concentration of the dose-enhancing agent.

6. A method according to claim 5, wherein the first set of CT data is acquired in conjunction with a first set of one or more scanning parameters, and further comprising:
associating the CT number and the sample concentration with one or more of the first set of one or more scanning parameters.

7. A method according to claim 6, wherein the one or more scanning parameters comprise at least one of:
an x-ray tube potential;
a scanning radiation energy;
an x-ray tube current;
a scan time;
slice width; and
radiation filtration parameters.

8. A method according to claim 6, further comprising:
acquiring a second set of CT data representing the phantom in conjunction with a second set of one or more scanning parameters, the second set being different from the first set; and
associating a CT number from the second set of CT data with the sample concentration of the dose-enhancing agent and with one or more of the second set of one or more scanning parameters.

9. A method according to claim 5, further comprising:
acquiring a second set of CT data representing a volume within a body, the volume including the dose-enhancing agent;
determining a CT number associated with the volume based on the second set of CT data; and
determining a concentration of the dose-enhancing agent within the volume based on the CT number associated with the volume and on the association between the CT number from the first set of CT data and the sample concentration of the dose-enhancing agent.

10. A method according to claim 9, wherein the first set of CT data is acquired in conjunction with a first set of one or more scanning parameters, wherein the second set of CT data is acquired in conjunction with a second set of one or more scanning parameters, and further comprising:
associating the CT number from the first set of CT data and the sample concentration with one or more of the first set of one or more scanning parameters,
wherein the step of determining the concentration comprises determining the concentration of the dose-enhancing agent within the volume based on the CT number associated with the volume, the second set of scanning parameters, and on the association between the CT number from the first set of CT data, the sample concentration of the dose-enhancing agent, and the first set of one or more scanning parameters.

11. A method according to claim 9, further comprising:
determining a dose enhancement based on the determined concentration.

12. A method according to claim 9, wherein the second set of CT data is acquired in conjunction with a first scanning radiation energy, further comprising:
acquiring, in conjunction with a second scanning radiation energy, a third set of CT data representing the volume within the body; and
determining a location of the dose-enhancing agent based on a difference between the second set of CT data and the third set of CT data.

13. A method according to claim 5, further comprising:
determining a dose enhancement associated with the sample concentration of the dose-enhancing agent; and
associating the dose enhancement with the CT number.

14. A method comprising:
acquiring, based on a first set of one or more CT scanning parameters, a first set of CT data representing a volume within a body including a dose-enhancing agent;
determining a CT number associated with the volume based on the first set of CT data; and
determining a dose enhancement corresponding to the dose-enhancing agent within the volume based on the CT number associated with the volume and on a stored association between the CT number, the one or more scanning parameters, and the dose enhancement.

15. A method according to claim 14, wherein the first set of CT data is acquired in conjunction with a first scanning radiation energy, further comprising:
acquiring, in conjunction with a second scanning radiation energy, a second set of CT data representing the volume within the body; and
determining a location of the dose-enhancing agent based on a difference between the first set of CT data and the second set of,CT data.

16. A method according to claim 15, further comprising:
presenting the difference between the first set of CT data and the second set of CT data to an operator.

17. A method according to claim 14, further comprising:
determining a concentration associated with the dose-enhancing agent within the volume based on the CT number associated with the volume and on an association between the concentration, the CT number, and the one or more scanning parameters.

18. A computer-readable medium storing computer-executable process steps, the process steps comprising:
a step to determine a CT number corresponding to each of a plurality of dose enhancements corresponding to a dose-enhancing agent for each of a plurality of sets of one or more CT scanning parameters.

19. A medium according to claim 18, the process steps further comprising:
a step to acquire a set of CT data representing a volume within a body, the volume including the dose-enhancing agent;
a step to determine a CT number associated with the volume based on the set of CT data; and
a step to determine a dose enhancement corresponding to the dose-enhancing agent within the volume based on the CT number associated with the volume and on the CT number corresponding to each of the plurality of dose enhancements for each of the plurality of sets of one or more CT scanning parameters.

20. A computer-readable medium storing computer-executable process steps, the process steps comprising:
a step to acquire a first set of CT data representing a phantom, the phantom comprising a dose-enhancing agent having a sample concentration; and
a step to associate a CT number from the first set of CT data with the sample concentration of the dose-enhancing agent.

21. A medium according to claim 20, wherein the first set of CT data is acquired in conjunction with a first set of one or more scanning parameters, the process steps further comprising:
a step to associate the CT number and the sample concentration with one or more of the first set of one or more scanning parameters.

22. A medium according to claim 21, the process steps further comprising:
a step to acquire a second set of CT data representing the phantom in conjunction with a second set of one or more scanning parameters, the second set being different from the first set; and
a step to associate a CT number from the second set of CT data with the sample concentration of the dose-enhancing agent and with one or more of the second set of one or more scanning parameters.

23. A medium according to claim 20, the process steps further comprising:
a step to acquire a second set of CT data representing a volume within a body, the volume including the dose-enhancing agent;
a step to determine a CT number associated with the volume based on the second set of CT data; and
a step to determine a concentration of the dose-enhancing agent within the volume based on the CT number associated with the volume and on the association between the CT number from the first set of CT data and the sample concentration of the dose-enhancing agent.

24. A medium according to claim 23, wherein the first set of CT data is acquired in conjunction with a first set of one or more scanning parameters, wherein the second set of CT data is acquired in conjunction with a second set of one or more scanning parameters, and the process steps further comprising:
a step to associate the CT number from the first set of CT data and the sample concentration with one or more of the first set of one or more scanning parameters,
wherein the step to determine the concentration comprises a step to determine the concentration of the dose-enhancing agent within the volume based on the CT number associated with the volume, the second set of scanning parameters, and on the association between the CT number from the first set of CT data, the sample concentration of the dose-enhancing agent, and the first set of one or more scanning parameters.

25. A medium according to claim 20, the process steps further comprising:
a step to determine a dose enhancement associated with the sample concentration of the dose-enhancing agent; and
a step to associate the dose enhancement with the CT number.

26. A computer-readable medium storing computer-executable process steps, the process steps comprising:
a step to acquire, based on a first set of one or more CT scanning parameters, a first set of CT data representing a volume within a body including a dose-enhancing agent;
a step to determine a CT number associated with the volume based on the first set of CT data; and
a step to determine a dose enhancement corresponding to the dose-enhancing agent within the volume based on a stored association between the CT number associated with the volume, the first set of one or more scanning parameters, and the dose enhancement.

27. An apparatus comprising:
a memory storing processor-executable process steps; and
a processor in communication with the memory and operative in conjunction with the stored process steps to:
determine a CT number corresponding to each of a plurality of dose enhancements corresponding to a dose-enhancing agent for each of a plurality of sets of one or more CT scanning parameters.

28. An apparatus according to claim 27, the processor further operative in conjunction with the stored process steps to:
acquire a set of CT data representing a volume within a body, the volume including the dose-enhancing agent;
to determine a CT number associated with the volume based on the set of CT data; and
to determine a dose enhancement corresponding to the dose-enhancing agent within the volume based on the CT number associated with the volume and on the CT number corresponding to each of the plurality dose enhancements for each of the plurality of sets of one or more CT scanning parameters.

29. An apparatus comprising:
a memory storing processor-executable process steps; and
a processor in communication with the memory and operative in conjunction with the stored process steps to:
acquire a first set of CT data representing a phantom, the phantom comprising a dose-enhancing agent having a sample concentration; and
associate a CT number from the first set of CT data with the sample concentration of the dose-enhancing agent.

30. An apparatus according to claim 29, wherein the first set of CT data is acquired in conjunction with a first set of one or more scanning parameters, the processor further operative in conjunction with the stored process steps to:
associate the CT number and the sample concentration with one or more of the first set of one or more scanning parameters.

31. An apparatus according to claim 29, the processor further operative in conjunction with the stored process steps to:
acquire a second set of CT data representing a volume within a body, the volume including the dose-enhancing agent;
determine a CT number associated with the volume based on the second set of CT data; and
determine a concentration of the dose-enhancing agent within the volume based on the CT number associated with the volume and on the association between the CT number from the first set of CT data and the sample concentration of the dose-enhancing agent.

32. An apparatus comprising:
a memory storing processor-executable process steps; and
a processor in communication with the memory and operative in conjunction with the stored process steps to:
acquire, based on a first set of one or more CT scanning parameters, a first set of CT data representing a volume within a body including a dose-enhancing agent;
determine a CT number associated with the volume based on the first set of CT data; and
determine a dose enhancement corresponding to the dose-enhancing agent within the volume based on a stored association between the CT number, the first set of one or more scanning parameters, and the dose enhancement.

33. A system comprising:
a surface to support a body;
a CT scanner to acquire, based on a first set of one or more CT scanning parameters, a first set of CT data representing a volume within the body including a dose-enhancing agent;
a processor to determine a CT number associated with the volume based on the first set of CT data, and to determine a dose enhancement corresponding to the dose-enhancing agent within the volume based on a stored association between the CT number, the first set of one or more scanning parameters, and the dose enhancement.

34. A system according to claim 33, wherein the first set of CT data is acquired in conjunction with a first scanning radiation energy,
the CT scanner to acquire, in conjunction with a second scanning radiation energy, a second set of CT data representing the volume within the body, and
the processor to determine a location of the dose-enhancing agent based on a difference between the first set of CT data and the second set of CT data.

35. A system according to claim 34, further comprising:
a display to present the location of the dose-enhancing agent to an operator.
